(19) United States
(12) Patent Application Publication
(10) Pub. No.: US 2025/0050075 A1
(43) Pub. Date: Feb. 13, 2025

(54) MEDICAL SHUNTS

(71) Applicant: THE BOARD OF TRUSTEES OF THE LELAND STANFORD JUNIOR UNIVERSITY, Stanford, CA (US)

(72) Inventors: Kunj Raju Sheth, San Francisco, CA (US); James K. Wall, Woodside, CA (US); Yair Blumenfeld, Palo Alto, CA (US); Eric Johnson, Woodside, CA (US); Enrico Danzer, Menlo Park, CA (US)

(21) Appl. No.: 18/233,204

(22) Filed: Aug. 11, 2023

(51) Int. Cl. A61M 27/00; A61M 25/00; A61M 25/04; A61M 39/24

(52) U.S. Cl. CPC A61M 27/002; A61M 25/0043; A61M 25/04; A61M 39/24; A61M 2025/0059; A61M 2210/145

(57) ABSTRACT

A medical device includes: an elongated member having a first end, a second end, and a body extending between the first end and the second end; a first anchor coupled to the first end of the elongated member; and a second anchor coupled to the second end of the elongated member; wherein the elongated member is configured for placement across an abdominal wall of a fetus, and wherein the elongated member is elastically stretchable to accommodate for a growing thickness of the abdominal wall of the fetus.

### RELATED APPLICATION DATA

This application is a continuation of International Patent Application No. PCT/US2022/015868 filed on Feb. 9, 2022, which claims priority to, and the benefit of, U.S. Provisional Patent Application No. 63/149,647 filed on Feb. 15, 2021, pending. The entire disclosures of the above applications are expressly incorporated by reference herein.

### FIELD

The field of the subject disclosure relates to medical devices, and more specifically medical shunts, such as vesicoamniotic shunts (VAS), thoraco-amniotic shunts, etc.

### BACKGROUND

Fetal lower urinary tract obstruction (LUTO), occurring in 2.2-3.4 of 10,000 live births, is most commonly due to posterior urethral valves (PUV) and urethral atresia.

While the severity of LUTO can vary, more severe fetal LUTO can lead to megacystis (markedly enlarged fetal bladder), bilateral hydronephrosis (dilated renal collecting system due to backward flow of fetal urine into the ureters and kidneys), in-utero renal injury due to high pressure destroying the nephrons, and anhydramnios (absence of amniotic fluid). Anhydramnios in turn results in irreversible pulmonary hypoplasia and pulmonary hypertension due to extrinsic compression of the uterine wall on the developing fetal chest. It is also thought that fetal swallowing of amniotic fluid assists with normal lung development although the precise mechanism of this remains unclear. Without in-utero therapy, LUTO is associated with an 80% mortality rate, mostly due to pulmonary injury incompatible with life.

Among survivors, neonatal and pediatric long-term renal morbidity remains significant, because the in-utero renal damage is irreversible. In the absence of fetal therapy, LUTO is routinely treated shortly after birth with diagnostic cystoscopy and valve ablation versus vesicostomy when treatment is not technically feasible. At present, about 62% of PUV cases are diagnosed on prenatal ultrasound, offering an opportunity to identify and treat fetuses in utero. While diversion of fetal urine into the amniotic cavity via a fetal VAS improves amniotic fluid volume and in-utero pulmonary development in severe LUTO patients, the effects of VAS on renal impairment remain. In addition, inherent limitations of existing shunts and the risks of performing an invasive procedure via the maternal abdomen limit fetal VAS to the most severe cases.

Despite the potential for improved survival, the risk for intraoperative and postoperative shunt complications is quite high. Shunt dislodgement, the most common complication, can occur in approximately 30-78% of cases. Because the shunt can migrate either completely into the fetal bladder or completely into the amniotic cavity over time, it is not uncommon for multiple procedures, sometimes as many as 4-5, to be necessary in a single pregnancy. Each invasive procedure also harbors a risk of premature rupture of membranes, chorion-amnion separation, placental trauma, trauma to fetal organs, fetal death, maternal amniotic fluid embolism, amnionitis leading to sepsis, premature labor and delivery, loss of the uterus or even death. In the Lucile Packard Children's Hospital (LPCH) experience, shunts have internalized completely within the fetal bladder or even the peritoneum, requiring surgical removal after birth. Alternatively, shunt migration out of the abdominal wall has led to implantation within the uterine wall or free-floating shunts in the amniotic cavity found during delivery. Even worse, there have been reports of shunt coils wrapping around a limb or umbilical cord compromising limb or life.

Although current VAS can be utilized for fetal treatment of LUTO, improving amniotic fluid volume and in-utero pulmonary development, the effects on renal impairment remain largely unknown. Furthermore, the inherent technical limitations of existing shunts and the risks of performing an invasive procedure via the maternal abdomen limit fetal therapy to the most severe LUTO cases. Furthermore, current VAS has been known to dislodge or migrate from its desired position relative to the patient after the VAS has been delivered.

### SUMMARY

Embodiments of shunt described herein address these current limitations with improved surgical specifications, reduced complications risks and a built-in mechanism to optimize in-utero bladder/renal function. Embodiments of shunt described herein may also improve survival as well as pulmonary and renal outcomes.

A medical device includes: an elongated member having a first end, a second end, and a body extending between the first end and the second end; a first anchor coupled to the first end of the elongated member; and a second anchor coupled to the second end of the elongated member; wherein the elongated member is configured for placement across an abdominal wall of a fetus, and wherein the elongated member is elastically stretchable to accommodate for a growing thickness of the abdominal wall of the fetus.

Optionally, the elongated member comprises a lumen extending from the first end to the second end.

Optionally, the elongated member is kink resistant.

Optionally, the elongated member comprises a coil, a braid, or a laser-cut tubing.

Optionally, the coil or the braid of the elongated member is made from an elastic material.

Optionally, the first anchor comprises a braided element.

Optionally, the braided element is configured to assume a delivery configuration when the braided element is contained in a delivery device, and to assume a deployed configuration when the braided element is outside the delivery device.

Optionally, the braided element is configured to expand radially when outside the delivery device.

Optionally, the braided element is made from Nitinol.

Optionally, the second anchor comprises a braided element.

Optionally, the first anchor comprises a plurality of leaflet elements.

Optionally, the leaflet elements are configured to assume a delivery configuration when the leaflet elements are contained in a delivery device, and to assume a deployed configuration when the leaflet elements are outside the delivery device.

Optionally, the medical device further includes a one-way valve coupled to the elongated member.

Optionally, the one-way valve is located in a lumen of the elongated member.

Optionally, the one-way valve is located at the first end or at the second end.

Optionally, the one-way valve is located closer to the first end than to the second end, or vice versa.

Optionally, the one-way valve is configured to open in response to pressure that is above a certain threshold.

Optionally, the medical device further includes a pressure sensor, wherein the one-way valve is configured to open based on input provided by the pressure sensor.

Optionally, at least a part of the medical device comprises echogenic enhancements.

A medical device includes: an elongated member having a first end, a second end, and a body extending between the first end and the second end; and a first anchor coupled to the first end of the elongated member; wherein the first anchor comprises a braided element; wherein the braided element is configured to assume a delivery configuration when the braided element is contained in a delivery device, and to assume a deployed configuration when the braided element is outside the delivery device.

Optionally, the first end is configured for placement inside a patient, and the second end is configured for placement outside the patient.

Optionally, the medical device further includes a second anchor coupled to the second end of the elongated member.

Optionally, the second end of the elongated member is configured to couple to a drainage container.

A medical device includes: an elongated member having a first end, a second end, and a body extending between the first end and the second end; a first anchor coupled to the first end of the elongated member; and a one-way valve located in a lumen of the elongated member.

Optionally, the medical device further includes a second anchor coupled to the second end of the elongated member.

Optionally, the first end is configured for placement inside a patient, and the second end is configured for placement outside the patient.

Optionally, the second end of the elongated member is configured to couple to a drainage container.

A medical device includes: an elongated member having a first end, a second end, and a body extending between the first end and the second end; and a first anchor coupled to the first end of the elongated member; wherein the elongated member is configured for placement between fluid from a first body cavity of a patient to a second body cavity of the patient or to a location outside the patient.

Other features will be described below in the detail description.

### BRIEF DESCRIPTION OF THE FIGURES

FIG. 1 illustrates a shunt and deliver device for delivering the shunt.

FIG. 2 illustrates a one-way valve that may be incorporated into the shunt of

FIG. 1.

FIG. 3 illustrates the shunt of FIG. 1, having been deployed to treat a fetus.

FIG. 4 illustrates a variation of the shut of FIG. 1.

FIGS. 5A-5C illustrate another variation of the shunt of FIG. 1.

FIG. 6 illustrates design features for the shunt for addressing different needs.

FIG. 7 illustrates alternative shunt solutions.

FIG. 8 illustrates testing setup and testing results for different shunts.

FIG. 9 illustrates a shunt flow test fixture.

FIG. 10 illustrates an apparatus for testing a shunt.

FIG. 11 illustrates the shunt device of FIG. 1 without any one-way valve.

FIG. 12 illustrates the shunt device of FIG. 1, particularly showing a variation of the design of the one-way valve.

FIGS. 13A-13E illustrate a technique of deploying the shunt of FIG. 1.

### DETAIL DESCRIPTION

Various embodiments are described hereinafter with reference to the figures. It should be noted that the figures may or may not be drawn to scale and that elements of similar structures or functions are represented by like reference numerals throughout the figures. It should also be noted that the figures are only intended to facilitate the description of the embodiments. They are not intended as an exhaustive description of the claimed invention or as a limitation on the scope of the claimed invention. In addition, an illustrated embodiment needs not have all the aspects or advantages of the invention shown. An aspect or an advantage described in conjunction with a particular embodiment is not necessarily limited to that embodiment and can be practiced in any other embodiments even if not so illustrated or if not so explicitly described.

In accordance with some embodiments, a medical device 10 (e.g., shunt) includes: an elongated member 20 having a first end 22, a second end 24, and a body 26 extending between the first end 22 and the second end 24; a first anchor 30 coupled to the first end 22 of the elongated member 20; and a second anchor 40 coupled to the second end 24 of the elongated member 20 (See FIG. 1). In the illustrated embodiments, the elongated member 20 is configured for placement across an abdominal wall of a fetus, or other fetal organ. The first anchor 30 is configured for placement inside a bladder of the fetus, and the second anchor 40 is configured for placement outside the fetus in the amniotic cavity. In such cases, the first anchor 30 may be considered as a bladder anchor, and the second anchor may be considered as an amniotic anchor. The anchors 30, 40 are made from braided Nitinol in the illustrated embodiments. In other embodiments, the anchors 30, 40 may have other form, and/or may be made from other materials.

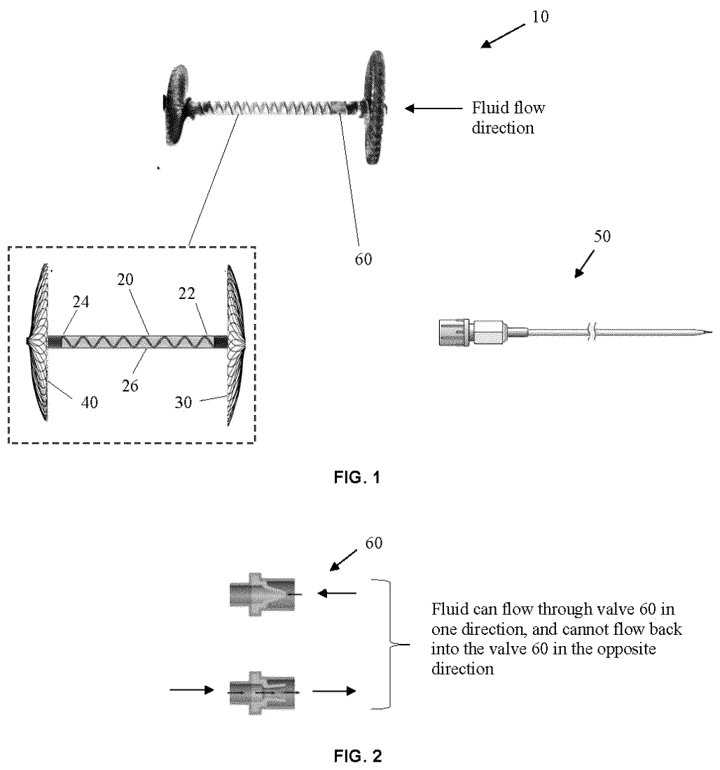

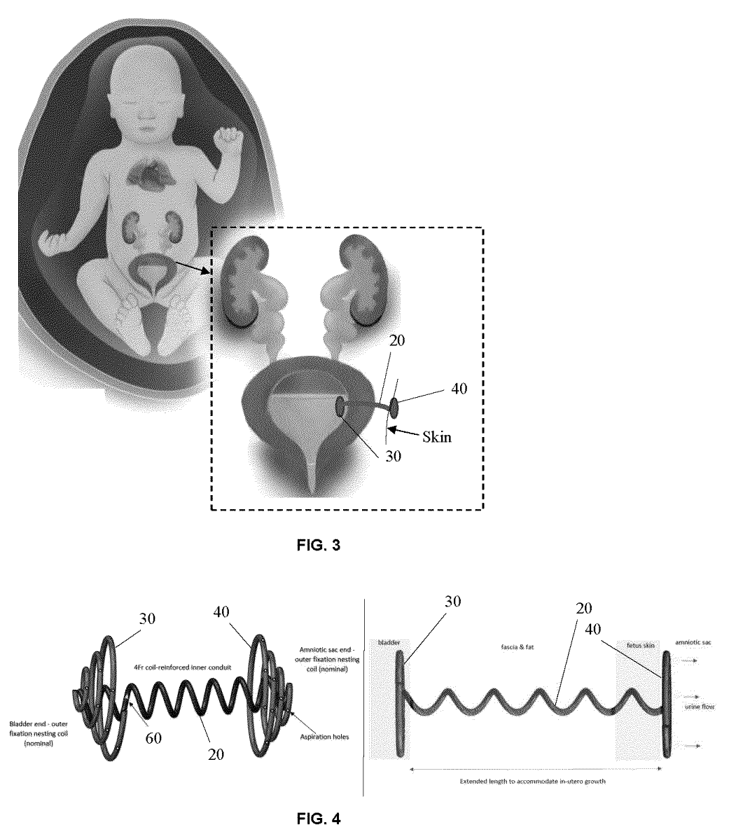

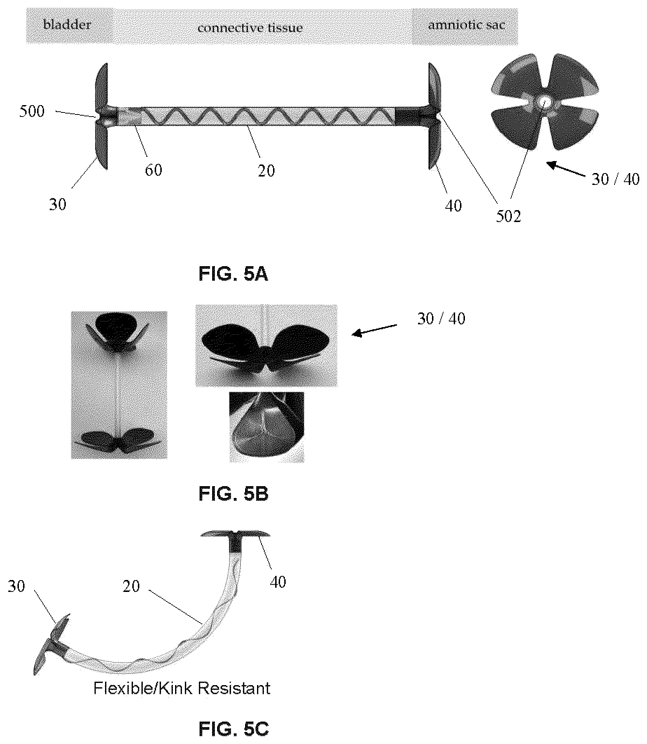

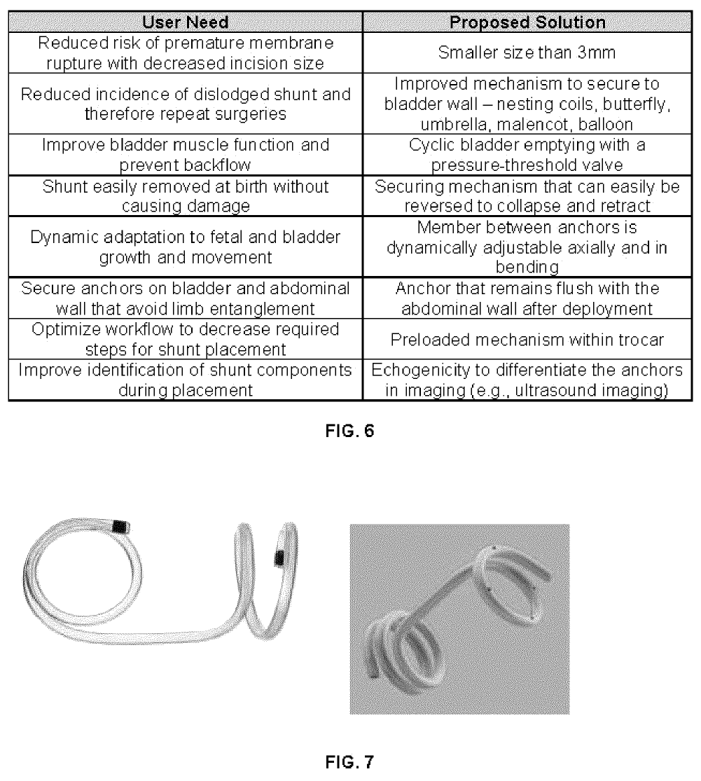

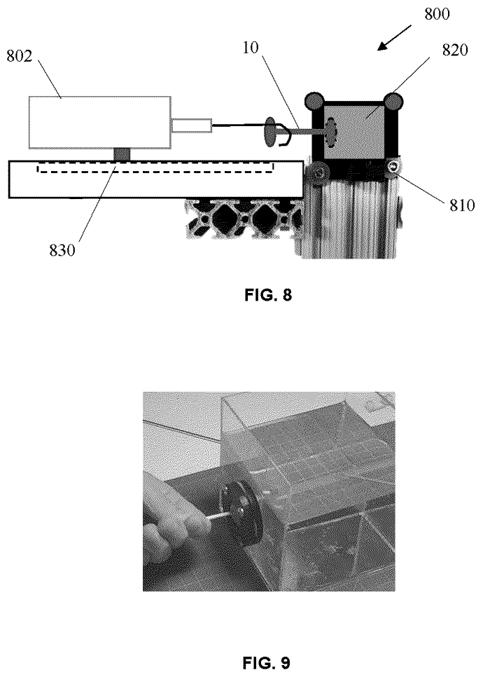

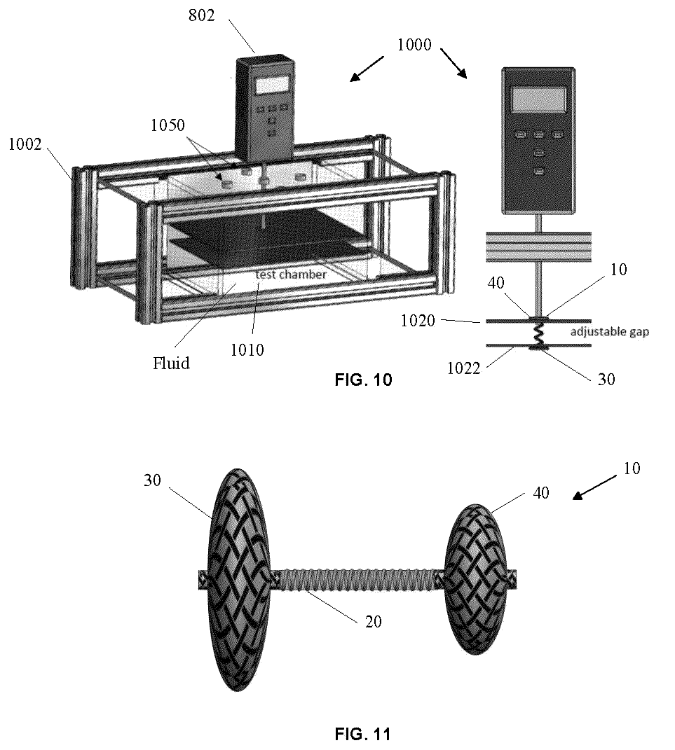

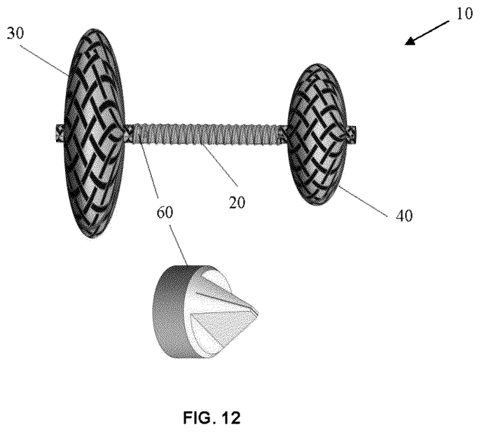

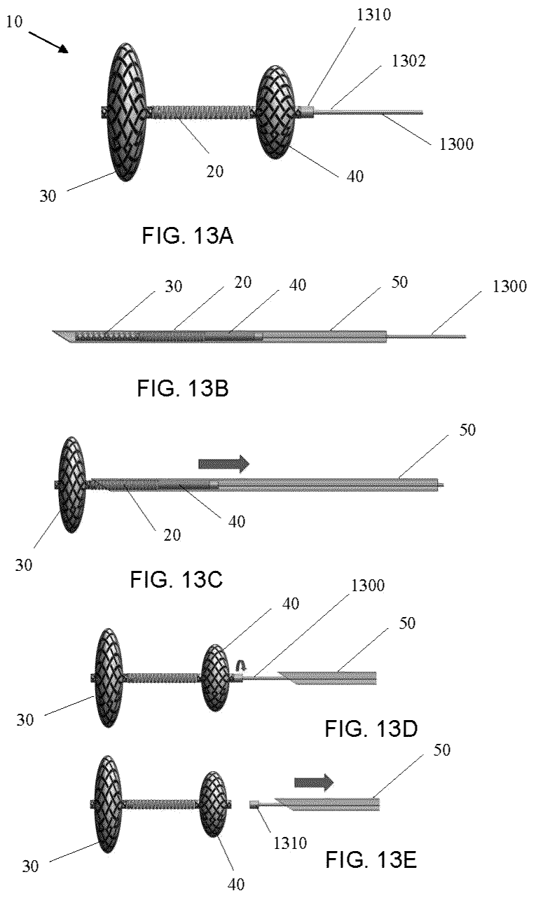

As shown in FIG. 2, in some embodiments, the shunt 10 may optionally further include a one-way valve 60 (e.g., a one-way duckbill valve made from silicone) coupled to the elongated member 20. The one-way valve 60 allows fluid (e.g., urine) to be transported from inside the patient to outside the patient, and also prevents the exited urine from flowing back to inside the patient. In some embodiments, the one-way valve 60 may be configured to simulate the urinating functionality and/or urinating schedule or urinating frequency of a human. The one-way valve 60 may be located in the elongated member 20. In some cases, the one-way valve 60 may be located closer to the anchor 30 than to the anchor 40. Alternatively, the one-way valve 60 may be located closer to the anchor 40 than to the anchor 30. In further embodiments, the one-way valve 60 may be located at a location that is the midpoint between the anchors 30, 40. In some embodiments, the one-way valve 60 is located at the first end 22 or at the second end 24. In other embodiments, the one-way valve 60 is located closer to the first end 22 than to the second end 24, or vice versa. In further embodiments, the one-way valve 60 may be located at the anchor 30 or at the anchor 40.

In some embodiments, the one-way valve 60 is configured to open in response to pressure that is above a certain threshold. The threshold may be selected to simulate a physiological pressure associated with bladder emptying. In some embodiments, the shunt 10 may further include a pressure sensor, wherein the one-way valve 60 is configured to open based on input provided by the pressure sensor. In some embodiments, the opening and/or closing of the valve 60 may be controlled by an external device that communicates with a switch at the valve. The communication may be achieved via a signal transmitter. The external device may be a computer, a cell phone, a hand-held controller, etc.

In some embodiments, instead of, or in addition to, the one-way valve 60, the shunt 10 may include any of other types of valves. In further embodiments, the shunt 10 may not include the one-way valve 60.

Also as shown in FIG. 1, the anchor 30 extends radially away from the elongated member 20. This feature also helps secure the anchor 30 inside the patient. Similarly, the anchor 40 extends radially away from the elongated member 20. This feature helps secure the anchor 40 outside the patient. In some embodiments, the anchors 30, 40 have the same cross-sectional dimension. In other embodiments, the anchor 30 may have a cross-sectional dimension that is larger than a cross-sectional dimension of the anchor 40. In further embodiments, the anchor 30 may have a cross-sectional dimension that is smaller than a cross-sectional dimension of the anchor 40.

In some embodiments, one or both of the expanded anchors 30, 40 may have a cross-sectional dimension that is anywhere from 4 mm to 20 mm, and more preferably, anywhere from 10 mm to 15 mm. In some cases, the expanded anchors 30, 40 may have the same cross-sectional dimension. In other cases, the expanded anchors 30, 40 may have different respective cross-sectional dimensions. For example, in some embodiments, the expanded anchor 30 may have a cross-sectional dimension that is anywhere from 12 mm to 16 mm (e.g., 14 mm), and the expanded anchor 40 may have a cross-sectional dimension that is anywhere from 8 mm to 11 mm (e.g., 10 mm).

In addition, as shown in the figure, the anchor 30 curves distally (e.g., away from the first end 22 of the elongated member 20 towards the second end 24). This way, at least a part of the anchor 30 lies in a plane intersecting the elongate member 20 at a location that is between the first end 22 and the second end 24. In other embodiments, the anchor 30 may curves proximally. In further embodiments, the anchor 30 may lie in a plane. In such cases, the anchor 30 does not curve distally and does not curve proximally. Similarly, as shown in the figure, the anchor 40 curves proximally (e.g., away from the second end 24 of the elongated member 20 towards the first end 22). This way, at least a part of the anchor 40 lies in a plane intersecting the elongate member 20 at a location that is between the first end 22 and the second end 24. In other embodiments, the anchor 40 may curves distally. In further embodiments, the anchor 40 may lie in a plane. In such cases, the anchor 40 does not curve distally and does not curve proximally.

As shown in FIG. 1, a delivery device 50 may be provided for delivery of the shunt 10. The delivery device 50 has a channel for housing the shunt 10. The shunt 10 has a delivery configuration inside the delivery device 50, in which the anchors 30, 40 (and optionally the elongated member 20) are stretched and/or bent to have a low profile. After the shunt 10 is delivered, the anchors 30, 40 will assume an expanded profile. In some embodiments, the delivery device 50 may optionally further include a Touhy Borst adaptor to assist delivery of the shunt 10.

During use, the shunt 10 is housed inside the delivery device 50, and the shunt 10 has a delivery configuration inside the delivery device 50. The delivery device 50 has a sharp distal tip 52 configured to pierce through a skin of the mother of the fetus, and through a wall of the uterus and the amniotic sac. In some embodiments, an insertion trocar may be utilized for insertion and/or placement of the delivery device 50. The delivery device 50 is also configured to pierce through an abdominal wall of the fetus, and through the detrusor muscle to access an inner space of the bladder of the fetus. The shunt 10 is then advanced distally relative to the delivery device 50, and/or the delivery device 50 is moved proximally relative to the shunt 10, until the first anchor 30 is outside the delivery device 50. Once outside the delivery device 50, the first anchor 30 expands radially to assume a deployed configuration. The first anchor 30 is configured to anchor against an inner wall of the bladder (FIG. 3). Next, the delivery device 50 is moved proximally to expose the elongated member 20, and to deploy the second anchor 40. In particular, the second anchor 40 is deployed outside the fetus (e.g., in the uterine cavity/amniotic fluid) so that the second anchor 40 anchors against a skin of the tetus (FIG. 3).

In some embodiments, ultrasound may be used to guide suprapubic placement of an insertion trocar. The shunt 10 may then be deployed. The placement of the shunt 10, and successful drainage of urine (from inside the bladder to outside the fetus) via the elongated member 20 may also be confirmed with ultrasound.

It should be noted that the shunt 10 is not limited to the above configuration, and that the shunt 10 may have other configurations in other embodiments. For example, in other embodiments, the first anchor 30 may have a spiral configuration, and the second anchor 40 may have a spiral configuration (FIG. 4). The elongated member 20 may also have a spiral configuration as shown in the figure. The spiral configuration of the elongated member 20 allows the elongated member 20 to change shape in correspondence to a varying distance between the anchors 30, 40. For example, the elongated member 20 may be stretched to accommodate movement of the uterus, and/or in-utero growth of the fetus. In some embodiments, the elongated member 20 may be kink-resistant while changing shape to increase the length along the longitudinal axis of the shunt 10.

In some embodiments, the elongated member 20 may have a length (e.g., total length) that is anywhere from 10 mm to 30 mm, and more preferably anywhere from 15 mm to 25 mm (e.g., 20 mm). In other embodiments, the elongated member 20 may have a length (e.g., total length) that is more than 30 mm, or less than 10 mm. Also, in some embodiments, the elongated member 20 may have a nominal length L (unstretched length), and may be stretchable to reach a total length that is at least: 1.2 L, or 1.3 L, or 1.4 L, or 1.5 L, or 1.6 L, or 1.7 L. For example, in some embodiments, the elongated member may have a nominal length of 20 mm, and may have an extended length of 30 mm.

In the illustrated embodiments shown in FIG. 4, the anchor 30 is a spiral tube with a lumen therein, wherein the lumen of the spiral tube is in fluid communication with a channel (conduit) in the elongated member 20. Similarly, the anchor 40 is a spiral tube with a lumen therein, wherein the lumen of the spiral tube is in fluid communication with the channel (conduit) in the elongated member 20. Each of the spiral tubes of the respective anchors 30, 40 has a plurality of holes. During use, urine from the bladder may enter into the holes at the spiral tube of the anchor 30. The urine goes through the one-way valve 60 inside the conduit of the elongated member 20 as the elongated member 20 transports the urine to the anchor 40. The urine then exits the openings at the spiral tube of the anchor 40.

As shown in the figure, the anchor 30 has a spiral configuration in which the member forming the anchor 30 has multiple turns (or loops) with different radius of curvatures. The elongated member 20 is connected to a part of the anchor 30 that has a relative smaller radius of curvature compared to another part of the anchor 30. This feature is advantageous as it may assist the anchor 30 in staying inside the patient so that the shunt 10 will not be dislodged. If the elongated member 20 is connected to the part of the anchor 30 that has the largest radius of curvature, the anchor 30 may be easily pulled out from the patient.

Similarly, the anchor 40 has a spiral configuration in which the member forming the anchor 40 has multiple turns (or loops) with different radius of curvatures. The elongated member 20 is connected to a part of the anchor 40 that has a relative smaller radius of curvature compared to another part of the anchor 40. This feature is advantageous as it may assist the anchor 40 in staying outside the patient. If the elongated member 20 is connected to the part of the anchor 40 that has the largest radius of curvature, at least a part of the anchor 40 may migrate inside the patient.

Also as shown in the figure, the spiral configuration of the anchor 30 has an increasing radius of curvature as so that the member forming the anchor 30 extends radially away from the elongated member 20. This feature also helps secure the anchor 30 inside the patient. Similarly, the spiral configuration of the anchor 40 has an increasing radius of curvature as so that the member forming the anchor 40 extends radially away from the elongated member 20. This feature helps secure the anchor 40 outside the patient.

In addition, as shown in the figure, the anchor 30 curves distally (e.g., away from the first end 22 of the elongated member 20 towards the second end 24). This way, at least a part of the anchor 30 lies in a plane intersecting the elongate member 20 at a location that is between the first end 22 and the second end 24. In other embodiments, the anchor 30 may curves proximally. In further embodiments, all of the loops of the anchor 30 may lie in a same plane. In such cases, the anchor 30 does not curve distally and does not curve proximally.

Similarly, as shown in the figure, the anchor 40 curves proximally (e.g., away from the second end 24 of the elongated member 20 towards the first end 22). This way, at least a part of the anchor 40 lies in a plane intersecting the elongate member 20 at a location that is between the first end 22 and the second end 24. In other embodiments, the anchor 40 may curves distally. In further embodiments, all of the loops of the anchor 40 may lie in a same plane. In such cases, the anchor 40 does not curve distally and does not curve proximally.

In the embodiments of FIG. 4, the anchors 30, 40 made be made from a material that is different from the material of the elongated member 20. In other embodiments, the anchors 30, 40 and the elongated member 20 may be made from the same material. In one implementation, a tubular structure may be utilized to form the elongated member 20 and the anchors 30, 40.

In further embodiments, the first anchor 30 may have a plurality of leaflet elements forming a “Tulip” (or “clover”) design, and the second anchor 40 may also have a plurality of leaflet elements (FIG. 5A). In the illustrated embodiments, the elongated member 20 is a tube with a thin wall made from an elastic material (e.g., silicone, PET, etc.). In some embodiments, the elongated member 20 may be implemented using a superelastic nitinol coil covered by a thin wall made from a stretchable material (e.g., silicone, PET, etc.). The shunt 10 also includes a first opening 500 at the first anchor 30, a second opening 502 at the second anchor 40, and a channel (conduit) in the elongated member 20 extending between the first opening 500 and the second opening 502.

The 4-leaf clover design of the anchor 30 is configured to anchor against the bladder, while the 4-leaf clover design of the anchor 40 is configured to anchor against the abdominal wall. In some embodiments, the anchor 30 and/or the anchor 40 may be made of ePTFE which has a different echogenicity due to its foam like material, allowing for accurate placement intraoperatively. In other embodiments, the anchors 30, 40 may be made from other materials. During use, the leaflets of the anchors 30, 40 are folded while the shut 10 is contained inside the delivery device 50. After the shunt 10 is deployed outside the delivery device 50, the leaflets of the anchors 30, 40 open up radially to assume respective expanded configurations. In some cases, the anchors 30, 40 may be folded into the trocar and then expand into the clover configuration after the shunt 10 is deployed.

In some embodiments, the anchors 30, 40 may have different shapes and/or sizes, which allow them to be differentiated in imaging. In other embodiments, the anchors 30, 40 may have the same shape and size. In such cases, different markers may be coupled to the respective anchors 30, 40 to allow them to be differentiated in imaging. In other embodiments, different portions of the respective anchors 30, 40 may have respective echogenicities of different shapes. This will also allow the anchors 30, 40 to be differentiated in imaging.

As shown in FIGS. 5A-5B, each of the anchors 30, 40 is the form of a leaflet system having four leaves. In other embodiments, each anchor 30/40 may have two or three leaves, or more than four leaves.

In some embodiments, the tulip design and the braid design may be more advantageous than the spiral design for the anchor 30 because they may be more resistant to dislodgement—i.e., such designs allow the anchor 30 to be more secured inside the bladder so that the anchor 30 will not come out of the patient by itself.

As shown in FIG. 5C, the elongated member 20 may be flexible in bending while being kink resistant. The elongated member 20 is also elastic in its axial direction to allow the elongated member 20 to undergo sufficient strain in order to accommodate fetus movement and/or growth of the fetus. The elongated member 20 may be made from a material and/or may include a reinforcing layer (e.g., a wire mesh, a coil, etc.), which provides the kink-resistant characteristic. In some embodiments, the elongated member 20 remains flexible while having the kink-resistant characteristic.

The shunt 10 is not limited to the above examples, and may have other features and/or combination of features.

In some embodiments, the elongated member 20 of the shunt 10 is elastically stretchable to accommodate for a growing thickness of the abdominal wall of the fetus.

In some embodiments, the elongated member 20 of the shunt 10 may be a coil element.

In some embodiments, the coil element may be configured to be delivered in an extended state, and contract upon deployment to bring two fluid communicating regions together/appose.

In some embodiments, the elongated member 20 of the shunt 10 may comprise a lumen extending from the first end 22 to the second end 24.

In some embodiments, the elongated member 20 of the shunt 10 may be kink resistant.

In some embodiments, the elongated member 20 of the shunt 10 may comprise a coil, a braid, or a laser-cut tubing.

In some embodiments, the coil or the braid of the elongated member 20 may be made from an elastic material (e.g., polymer, metal, alloy, etc.).

In some embodiments, the coil or the braid of the elongated member 20 may be made from Nitinol.

In some embodiments, the elongated member 20 of the shunt 10 may be a coil reinforced member, and may include a channel for transportation of fluid. In some embodiments, the elongated member 20 may be a tube made from PET, and may have a reinforcement layer in the tube, wherein the reinforcement layer may be made from Nitinol.

In some embodiments, the elongated member 20 of the shunt 10 may be biocompatible.

In some embodiments, the elongated member 20 may be 4 Fr, 5 Fr, 6 Fr, or any of other sizes.

In some embodiments, the elongated member 20 of the shunt 10 may be flexible in the bending and axial direction, while being kink-resistant.

In some embodiments, the elongated member 20 of the shunt 10 may have a memory shape. In such cases, when the elongated member 20 of the shunt 10 is contained in the delivery device 50, the elongated member 20 is stretched/compressed into a low profile. When the elongated member 20 is delivered outside the delivery device 50, the elongated member 20 assumes its delivery shape (memory shape).

In some embodiments, the first anchor 30 of the shunt 10 may comprise a braided element.

In some embodiments, one or both of the anchors 30, 40 of the shunt 10 may be partially or completely covered with an impermeable, biocompatible element.

In some embodiments, one or both of the anchors 30, 40 of the shunt 10 may be atraumatic.

In some embodiments, one or both of the anchors 30, 40 of the shunt 10 may be echogenic.

In some embodiments, one or both of the anchors 30, 40 of the shunt 10 may be made from a foam.

In some embodiments, one or both of the anchors 30, 40 may of the shunt 10 be made from ePTFE.

In some embodiments, one or both of the anchors 30, 40 may include leaflets made from a foam, and supports (for the leaflets) made from FEP.

In some embodiments, the anchor 30/40 may extend radially away from the elongated member 20. The radial extension may be in one direction, or in multiple directions.

In some embodiments, the anchor 30/40 may have a dome shape.

In some embodiments, the shunt 10 may be coated with a pharmacological agent.

In some embodiments, some or all shunt 10 components may be biodegradable.

In some embodiments, the braided element of the anchor 30/40 is configured to assume a delivery configuration when the braided element is contained in the delivery device 50, and to assume a deployed configuration when the braided element is outside the delivery device 50.

In some embodiments, the braided element is configured to expand radially when outside the delivery device 50.

In some embodiments, the braided element is made from an elastic material (e.g., polymer, metal, alloy, etc.).

In some embodiments, the second anchor 40 of the shunt 10 may comprise a braided element.

In some embodiments, one or each of the first and second anchors 30, 40 of the shunt 10 has a plurality of fluid communicating regions.

In some embodiments, one or each of the first and second anchors 30, 40 of the shunt 10 may have concave or convex.

In some embodiments, the first anchor 30 of the shunt 10 may comprise a plurality of leaflet elements.

In some embodiments, the leaflet elements are configured to assume a delivery configuration when the leaflet elements are contained in the delivery device 50, and to assume a deployed configuration when the leaflet elements are outside the delivery device 50.

In some embodiments, at least a part of the shunt 10 may be echogenic (e.g., at least a part of the shunt 10 may incorporate echogenic enhancements).

In some embodiments, the delivery device 50 may be coupled to the shunt 10 to facilitate positioning, controlled release of the shunt 10, and recapturing of the shunt 10. The coupling may be threaded, magnetic, electronically actuated, or other mechanical coupling.

In some embodiments, the delivery device 50 may be a 4 Fr, 5 Fr, 6 Fr, 7 Fr, or 8 Fr delivery device.

In some embodiments, a medical system may include the shunt 10 and an insertion trocar.

In some embodiments, there may be different insertion trocars with different respective sizes.

In some embodiments, there may be different shunts 10 with different respective sizes.

In some embodiments, there may be different delivery devices 50 with different respective sizes, wherein the different delivery devices 50 are configured to deliver different shunts 10 with different respective sizes.

In some embodiments, one or more of the below features of the shunt 10 may be optimized: 1) anchoring features that maintain the positional stability of the shunt 10 tor the duration of the implant, 2) shunt conduit through which fetal urine will pass, 3) a one-way valve to maintain a pre-determined bladder pressure and allow urine to pass once that pressure is exceeded, and 4) the delivery system and method.

The shunt 10 may be made from a variety of materials. In some embodiments, at least a part of the first anchor 30, at least a part of the second anchor 40, and/or at least a part of the elongated member 20, may be made from Nitinol, such as Nitinol wire (NDC Freemont, CA), Nitinol Coils/braids (Kellogg Research Group Nashua, NH), etc. The Nitinol material allows the shunt 10 to be visualized via imaging (e.g., ultrasound) during delivery of the shunt 10. In addition, in some embodiments, the elongated member 20 may be made from polymeric extrusion(s) (Duke Empirical Santa Cruz, CA). In some cases, polymer(s) may be applied onto the elongated member 20 by coating the elongated member 20 in an elastic polymer (e.g. polyurethane or other biocompatible elastomer), by covering the elongated member 20 with a polymer incorporating pleats (which allow for axial expansion), or by a combination of the above techniques.

In the embodiments in which the anchors 30, 40 comprise respective braids, the braided anchors 30, 40 are advantageous because they can assume a low profile while contained inside the delivery device 50, and can expand radially to form expanded anchors 30, 40. The low profile of the braid anchor also prevents the fetus from grasping and/or dislodging the shunt 10. The braided anchor 30 inside the fetus is also advantageous because the openings and porosity of the braided anchor 30 allows fluid to enter into the anchors 30 (like a storm drain tubing), and be received by the lumen inside the elongated member 20. Furthermore, the axially adjustable feature of the elongated member 20 is advantageous because it allows the delivered shunt 10 to accommodate with the growing geometry and movement of the fetus. Also, the echogenicity feature of the shunt 10 allows it to be positioned and delivered efficiently and accurately. In addition, the one-way valve 60 is beneficial because it prevents fluid drained from the bladder of the fetus from entering back into the bladder.

In some embodiments, the shunt 10 may be designed according to one or more of the user's needs summarized in the left column of FIG. 6. The shunt 10 may have one or more of the features stated in the right column of FIG. 6. For example, the shunt 10 may be designed to have a size smaller than existing solutions, in order to reduce risk of premature membrane rupture, and to decrease incision size. For example, in some embodiments, the shunt 10 may have a size that is less than 3 mm, less than 2 mm, less than 1 mm, etc., when being delivered by the delivery device 50.

As another example, the shunt 10 may have a securing mechanism designed to secure to an interior bodily region (e.g., bladder wall) of a patient. The securing mechanism may include one or more coils (e.g., nesting coils), a balloon, an anchor with a butterfly-configuration, an anchor with an umbrella configuration, a malecot, etc. In some embodiments, the securing mechanism may be configured so that the anchor pull-out force is higher than 0.35 lbf, higher than 0.4 lbf, higher than 0.5 lbf, higher than 0.6 lbf, higher than 0.7 lbf, higher than 0.8 lbf (e.g., 0.84 lb.), etc. In some embodiments, the shunt 10 may have a pull-out force that is more than 2 times, more than 4 times, more than 10 times, or more than 20 times than that of another shunt solution. For example, another shunt solution has a pigtail coil or a double pigtail coil that can easily be pulled out.

As a further example, the shunt 10 may include a one-way valve configured to allow cyclic bladder emptying based on a pressure threshold. This feature may improve bladder muscle function, and may prevent backflow of urine.

As another example, the shunt 10 may be designed to have a securing mechanism that can easily be reversed to collapse the shunt 10, and to retract the shunt 10. For example, at birth, the shunt 10 can be easily removed without causing damage to the baby. In one implementation, a retriever may be coupled to the shunt 10, and may retract the shunt 10 into a sheath. The retriever may have a coupler (e.g., a threaded attachment) configured to attach to an end of the shunt 10.

As another example, the elongated member 20 may be designed to have a variable length, so that the elongated member 20 can change length in-vivo. This allows the fetus to move and to grow without interfering with the functionality of the shunt 10.

As a further example, the anchor 40 may have a deployed shape that remains flush with the abdominal wall of the fetus after deployment. This feature avoids limb entanglement, and may prevent the fetus from pulling out the shunt 10.

In another example, the shunt 10 may be preloaded in a trocar. This may optimize workflow to decrease the number of steps required for placement of the shunt 10.

In another example, the shunt 10 may be echogenic, which allows one or more components of the shunt 10 to be visualized by imaging. For example, the anchors 30, 40, and/or the elongated member 20 may be echogenic in some embodiments.

Also, in some embodiments, the shunt 10 may be echogenic so that it is viewable via medical imaging. For example, at least a part of the elongated member 20, at least a part of the anchor 30, at least a part of the anchor 40, or any combination of the foregoing, may be made from a material that is echogenic.

It should be noted that the shunt 10 is not required to have all of the above features described with reference to FIG. 6, and that the shunt 10 may have only one, or selected ones of the features.

It should be noted that the embodiments of the shunt 10 described herein are significant improvements over the pigtail design or the double pigtail coils shown in FIG. 7. In the designs of FIG. 7, the shunts do not have dynamically adjustable elongated member that accommodates a growth of the fetus. The shunts of FIG. 7 also do not have any one-way pressure valve to facilitate in-utero intermittent bladder cycling allowing the development of normal bladder contractility. Furthermore, the shunts of FIG. 7 have less pull-out force—which means that the shunts can be easily pulled out.

In some embodiments, an apparatus may be provided to perform force testing for the shunt 10. FIG. 8 illustrates an example of such apparatus 800. The apparatus 800 includes a force gauge 802 configured to determine how much force is being applied on the shunt 10. The apparatus 800 also includes a tissue mount 810 configured to mount an artificial tissue, such as a porcine bladder 820 (available at Animal Technologies, Tyler TX). The porcine bladder 820 is configured to simulate a neonatal bladder. As shown in the figure, the force gauge 802 is supported on bearing 830 so that the force gauge 802 is moveable relative to the tissue mount 810 (and relative to the porcine bladder 820 secured by the tissue mount 810). During use, the porcine bladder 820 is secured at the tissue mount 810. The shunt 10 is then loaded and the anchor 30 of the shunt 10 is deployed into the porcine bladder 820 to simulate an interaction between the shunt 10 and a bladder. The force gauge 802 is attached to the proximal end of the shunt 10, and the force gauge 802 is translated on the bearing 830. The movement of the force gauge 802 pull the proximal end of the shunt 10 proximally, thereby applying force onto the shunt 10. As the proximal end of the shunt 10 is moved further proximally, the force applied onto the shunt 10 increases. Initially, when the force is low, the anchor 30 stays inside the porcine bladder 820. When sufficient force is applied onto the shunt 10, the anchor 30 of the shunt 10 will be pulled out from the porcine bladder 820.

In some embodiments, a model bladder test chamber may be provided to test the patency of the shunt design iterations and characterize release pressure using a porcine bladder tissue mounted to an open-manometer pressure chamber (FIG. 9). As shown in the figure, the model bladder test chamber includes a circular frame holding a layer of porcine bladder tissue. The model bladder test chamber also includes a tank modeling the bladder, and water in the tank modeling urine. This testing fixture may be utilized to test the valve mechanism (e.g., the one-way valve 60) of the shunt 10, enabling bladder cycling of urine in a fashion similar to normal non-obstructed bladders. The bladder cycling will allow early and regular use of the detrusor muscle, preventing it from undergoing fibrosis and ultimately myogenic failure postnatally.

Refer now to FIG. 10, in some embodiments, another apparatus 1000 (e.g., a benchtop test fixture) may be provided to test a shunt (e.g., the shunt 10). The apparatus 1000 includes a frame 1002 (e.g., made from aluminum or any of other materials), a test chamber 1010, a first layer 1020 of material for simulating a patient's skin, and a second layer 1022 of material for simulating a bladder. The apparatus 1000 further includes a force gauge 802. The force gauge is configured to measure an amount of force being exerted on the shunt 10. The chamber 1010 is configured to test the performance of the one-way valve of the shunt 10. In particular, the chamber 1010 is sealed during use, and may enable a variety of bladder pressures to be simulated. In some cases, the chamber 1010 may be utilized in a process to characterize and optimize the performance of the one-way valve of the shunt 10. The apparatus 1000 optionally further includes a plurality of indexing holes 1050 at the top of the test chamber, which allow the force testing to be performed on the shunt at a number of different displacement force angles (e.g. 0°, 15°, 30°, 45═). In some cases, the apparatus 1000 may optionally further include a valve configured to adjust a pressure of the fluid contained inside the chamber 1010.

During use of the apparatus 1000, the layers 1020, 1022 simulating the skin and bladder interfaces are contained inside the chamber 1010. Fluid simulating bladder pressure is also placed inside the chamber 1010. The force gauge 802 grasps one end of the shunt 10. In some embodiments, the force gauge 802 made be moved relative to the layer 1022 (against which the anchor 30 is anchored), or the layer 1022 may be moved relative to the force gauge 802, to increase the force being applied to the shunt 10. The force is increased until the anchor 30 is pulled out from one side of the layer 1022 to the other side of the layer 1022. The force may then be recorded as the pull-out force for the shunt 10. Also, in some embodiments, the distance between the layers 1020, 1022 may be adjusted to simulate a movement of a patient, and/or to simulate a growing fetus. This allows the elongated member 20 to be tested to see if it can change size and/or shape to accommodate the growth and/or movement of a fetus. Furthermore, the fluid pressure in the chamber allows the one-way valve of the shunt 10 to be tested, and see if the valve will allow fluid on one side of the layer 1022 (simulating the bladder interface) to pass therethrough, and to exit via opening(s) at the anchor 40.

It should be noted that the shunt 10 is not required to include the one-way valve 60. For example, as shown in FIG. 11, in some embodiments, the shunt 10 may not include the one-way valve 60.

Also, in the embodiments in which the shunt 10 includes the one-way valve 60, the one-way valve 60 may have different designs in different embodiments. Thus, the one-way valve 60 is not required to have the configuration shown in FIG. 2. For example, in some embodiments, the one-way valve 60 may have the configuration shown in FIG. 12. In the illustrated embodiments, fluid flow in one direction (from left to right) will force the flaps of the valve 60 to open, thereby allowing fluid to pass through the valve 60. On the other hand, fluid flow in the opposite direction (from right to left) will force the flaps pf the valve 60 to close, thereby preventing fluid from passing through the valve 60.

FIG. 13A-13E illustrate a technique of deploying the shunt 10. As shown in FIG. 13A, a pusher 1300 in a form of a rod has a distal end 1302 with a coupler 1310 configured to couple to an end of the shunt 10. The shunt 10 is contained in the delivery device 50 (e.g., a sheath, a cannula, or any tubular structure) before the shunt 10 is deployed (FIG. 13B). When the shunt 10 is contained in the delivery device 50, the anchors 30, 40 is compressed and/or stretched to have a low profile. The delivery device 50 may have a sharp distal tip for piercing tissue. During use, the delivery device 50 may be utilized to pierce through a skin of a patient carrying a fetus, through the wall of the uterus, through a skin of a fetus, and through a wall of the bladder to reach an inside of the bladder of the fetus. Then the pusher 1300 is advanced relative to the delivery device 50, and/or the delivery device 50 is retracted relative to the pusher 1300, so that the anchor 30 is deployed outside the delivery device 50 into the interior of the bladder of the fetus (see FIG. 130, and FIG. 3). Next, the delivery device 50 is retracted relative to the pusher 1300, so that the elongated member 20 is deployed outside the delivery device 50, to a position that is across the wall of the bladder and across a skin of the fetus. The delivery device 50 is then further retracted relative to the pusher 1300, so that the anchor 40 is deployed outside the delivery device 50, to a position that is outside the fetus (see, for example, FIG. 3). After the anchor 40 is deployed, the pusher 1300 may be operated (e.g., twisted) to detach the coupler 1310 from the shunt 10 (FIG. 130). The delivery device 50 and the pusher 1300 may then be retracted to remove from the ulerus, and from the patient carrying the fetus.

It should be noted that the embodiments of the shunt 10 described herein are not limited for treating LUTO, and are not limited to being placed across the abdominal wall of the fetus. In other embodiments, the shunt 10 may be utilized to treat other conditions, and may be placed in any locations inside a patient.

For example, in other embodiments, the shunt 10 may be used as a thoraco-amniotic shunt for the treatment of fetal pleural effusions, fetal hydrops, or macrocystic congenital lung lesions. Similar complications in the currently known treatment can arise including shunt dislodgment and fetal chest trauma Therefore, percutaneous fetal lung shunt procedures are also reserved for the most severe cases in which extra fluid or a mass within the fetal thoracic cavity is large enough to cause mediastinal shift and organ compression. Thus, embodiments of the shunt described herein may be used in the lungs or other areas in a patient to treat any fetal conditions. For fetal pleural effusion/hydrothorax, one end of the shunt described herein will be placed in a fetal lung. For large fetal lung masses, one end of the shunt described herein will be placed in the fetal lung. Also, for fetal ascites, one end of the shunt described herein will be placed in the fetal abdomen.

It should be noted that embodiments of the shunt 10 described herein are not limited to treat conditions in fetus, and may be used to treat conditions in babies, children, and adults.

Also, embodiments of the shunt 10 described herein may be used to treat any condition where a fluid collection in a patient needs to be drained or diverted. For example, in some embodiments, the shunt 10 may be configured for use as a cardiovascular bypass graft, or an arteriovenous graft for hemodialysis. In addition, in some embodiments, one end (e.g., the first end) of the shunt 10 may be placed inside the body of the patient, and the other end (e.g., the second end) of the shunt 10 may be placed outside the body of the patient. Also, in some embodiments, the second end of the shunt 10 outside the patient's body may be configured to couple to a drainage container (e.g., collection bag, bottle, sampling tube, etc.). Furthermore, in some embodiments, the elongate member 20 of the shunt 10 may be a catheter with one end (e.g., the first end) coupled to the first anchor 30 for placement inside a patient, and the other end (e.g., the second end) being for placement outside the patient. In further embodiments, the elongated member 20 may be configured to couple to a catheter or to a tube. For example, in some embodiments, the second end 24 of the elongated member 20 may be coupled to a tube, wherein the coupling may be permanent, or may be a detachable coupling.

Furthermore, in other embodiments, the shunt 10 may be used to drain or transport fluid from a first body cavity of a patient to a second body cavity of the patient. In such cases, both anchors 30, 40 are configured for placement inside the patient. The first body cavity may be any cavity inside the patient, and so it is not limited to a bladder cavity.

Although particular embodiments have been shown and described, it will be understood that it is not intended to limit the claimed inventions to the preferred embodiments, and it will be obvious to those skilled in the art that various changes and modifications may be made without department from the scope of the claimed inventions. The specification and drawings are, accordingly, to be regarded in an illustrative rather than restrictive sense. The claimed inventions are intended to cover alternatives, modifications, and equivalents.

1. A medical device, comprising:
an elongated member having a first end, a second end, and a body extending between the first end and the second end;
a first anchor coupled to the first end of the elongated member; and
a second anchor coupled to the second end of the elongated member;
wherein the elongated member is configured for placement across an abdominal wall of a fetus, and wherein the elongated member is elastically stretchable to accommodate for a growing thickness of the abdominal wall of the fetus.

2. The medical device of claim 1, wherein the elongated member comprises a lumen extending from the first end to the second end.

3. The medical device of claim 1, wherein the elongated member is kink resistant.

4. The medical device of claim 1, wherein the elongated member comprises a coil, a braid, or a laser-cut tubing.

5. The medical device of claim 1, wherein the first anchor comprises a braided element.

6. The medical device of claim 5, wherein the braided element is configured to assume a delivery configuration when the braided element is contained in a delivery device, and to assume a deployed configuration when the braided element is outside the delivery device.

7. The medical device of claim 6, wherein the braided element is configured to expand radially when outside the delivery device.

8. The medical device of claim 1, wherein the first anchor comprises a plurality of leaflet elements.

9. The medical device of claim 8, wherein the leaflet elements are configured to assume a delivery configuration when the leaflet elements are contained in a delivery device, and to assume a deployed configuration when the leaflet elements are outside the delivery device.

10. The medical device of claim 1, further comprising a one-way valve coupled to the elongated member.

11. The medical device of claim 10, wherein the one-way valve is located in a lumen of the elongated member.

12. The medical device of claim 10, wherein the one-way valve is located closer to the first end than to the second end, or vice versa.

13. The medical device of claim 10, wherein the one-way valve is configured to open in response to pressure that is above a certain threshold.

14. The medical device of claim 10, further comprising a pressure sensor, wherein the one-way valve is configured to open based on input provided by the pressure sensor.

15. The medical device of claim 1, wherein at least a part of the medical device comprises echogenic enhancements.

16. The medical device of claim 1, wherein the first anchor is configured for placement inside a patient, and the second anchor is configured for placement outside the patient.

17. The medical device of claim 16, wherein the elongated member is configured to couple to a drainage container outside the patient.

18. A medical device, comprising:
an elongated member having a first end, a second end, and a body extending between the first end and the second end;
a first anchor coupled to the first end of the elongated member; and
a one-way valve located in a lumen of the elongated member.

19. The medical device of claim 18, further comprising a second anchor coupled to the second end of the elongated member.

20. The medical device of claim 18, wherein the first end is configured for placement inside a patient, and the second end is configured for placement outside the patient.
